SEQUENCE LISTING (10 sequences)

SEQ ID NO: 1 - DNA, 34 bp, Artificial sequence
  source 1..34: /note= “Description of artificial sequence Primer Pr1”
ctgatcagttcgtgtccgtacaactggcgtaatc

SEQ ID NO: 2 - DNA, 34 bp, Artificial sequence
  source 1..34: /note= “Description of artificial sequence Primer Pr2”
atacgctgtattcagcaacaccgtcaggaacacg

SEQ ID NO: 3 - DNA, 34 bp, Artificial sequence
  source 1..34: /note= “Description of artificial sequence Primer Pr3”
ccagccgcaatatctggcggtgcaatatcggtac

SEQ ID NO: 4 - DNA, 34 bp, Artificial sequence
  source 1..34: /note= “Description of artificial sequence Primer Pr4”
gtgcaccatgcaacatgaataacagtgggttatc

SEQ ID NO: 5 - DNA, 33 bp, Artificial sequence
  source 1..33: /note= “Description of artificial sequence Primer Pr5”
gaaagttatccctagtcagtggcctgaagagac

SEQ ID NO: 6 - DNA, 32 bp, Artificial sequence
  source 1..32: /note= “Description of artificial sequence Primer Pr6”
taatgcaaactacgcgccctcgtatcacatgg

SEQ ID NO: 7 - DNA, 18 bp, Artificial sequence
  source 1..18: /note= “Description of artificial sequence Primer PrCSFR1”
cctggagctgagaactac

SEQ ID NO: 8 - DNA, 18 bp, Artificial sequence
  source 1..18: /note= “Description of artificial sequence Primer PrCSFR2”
tcccggctgagttatagg

SEQ ID NO: 9 - DNA, 26 bp, Artificial sequence
  source 1..26: /note= “Description of artificial sequence Primer Pr-RT1”
cctgaaccacccaggccagattggtg

SEQ ID NO: 10 - DNA, 26 bp, Artificial sequence
  source 1..26: /note= “Description of artificial sequence Primer Pr-RT2”
gagggtagtcagagaagctctccacg

(19) United States
(12) Patent Application Publication
(10) Pub. No.: US 2009/0170090 A1
(43) Pub. Date: Jul. 2, 2009

(54) Method for Enhancing Enzymatic DNA Polymerase Reactions

(75) Inventors: Konstantin Ignatov, Moskau (RU); Vladimir Kramarov, Moskau (RU); Dimitrij Plachov, Münster (DE)

Correspondence Address: NIXON & VANDERHYE, PC, 901 NORTH GLEBE ROAD, 11TH FLOOR, ARLINGTON, VA, 22203, US

(73) Assignee: BIOLINE LIMITED, London (GB)

(21) Appl. No.: 12/085,159

(22) PCT Filed: Nov. 15, 2006
(86) PCT No.: PCT/EP2006/010951

§ 371 Date: Oct. 3, 2008

Related U.S. Application Data
(60) Provisional application No. 60/738,407, filed on Nov. 18, 2005.

(51) Int. Cl. C12Q 1/68; C12P 19/34

(52) U.S. Cl. 435/6; 435/91.2

(57) ABSTRACT

The invention relates to a method of enhancing a DNA polymerase reaction by including in a reaction mixture containing a DNA polymerase a protein of DNA ligase.

### FIELD OF THE INVENTION

The present invention relates to improvements in methods of enzymatic synthesis and amplifications of DNA. The invention provides methods, kits, proteins and compositions of proteins for enhancing enzymatic DNA polymerase reactions.

The disclosed methods of the invention are in particularly useful for improving polymerase chain reaction (PCR), and particularly useful for enhancing yield of a long distance PCR and/or a low copy DNA template PCR amplification. The invention also can be useful for improving other laboratory procedures using DNA polymerases, such as primer extension, reverse transcription and DNA sequencing.

### BACKGROUND ART

The present invention is aimed at increasing the efficiency of nucleic acid polymerization reactions by a novel formulation of enzymes, and more particularly, the efficiently of catalysing the amplification by PCR of long and low copy DNA templates.

Reactions of template-directed polymerization of deoxyribonucleoside triphosphates (dNTPs) to form DNA are used in a variety of in vitro DNA synthesis applications, such as primer extension techniques, DNA sequencing and DNA amplification. Manipulating DNA by means of polymerization reactions is a fundamental component of biotechnology-related research.

DNA polymerases, the enzymes which catalyze DNA polymerization reactions, are well known, and are useful in a wide range of laboratory processes, especially in molecular biology. Thermostable DNA polymerases have benefits in a number of techniques, as thermostable enzymes can be used at relatively high temperatures. Thermostable DNA polymerases are particularly useful in polymerase chain reaction (PCR).

PCR is very important for the development of the biotechnology industry as well as for basic biological research. PCR reactions today are carried out by the use of a heat-resistant DNA polymerase enzyme (such as Taq DNA polymerase) in a multi-cycle process employing several alternating heating and cooling steps to amplify the DNA (U.S. Pat. Nos. 4,683,202 and 4,683,195). First, a reaction mixture is heated to a temperature sufficient to denature the double stranded target DNA into its two single strands. The temperature of the reaction mixture is then decreased to allow specific oligonucleotide primers to anneal to their respective complementary single-stranded target DNA. Following the annealing step, the temperature is raised to the temperature optimum of the DNA polymerase being used, which allows incorporation of complementary nucleotides at the 3′ ends of the annealed oligonucleotide primers thereby recreating double stranded target DNA. Using a heat-stable DNA polymerase, the cycle of denaturing, annealing and extension may be repeated as many times as necessary to generate a desired product, without the addition of polymerase after each heat denaturation. Twenty or thirty replication cycles can yield up to a million-fold amplification of the target DNA sequence (“Current Protocols in Molecular Biology,” F. M. Ausubel et al. (Eds.), John Wiley and Sons, Inc., 1998).

DNA polymerases obtained from bacteria of genus “Thermus”, such as Thermus aquaticus (Taq DNA polymerase) [U.S. Pat. No. 4,889,818 and No. 5,079,352], Thermus flavus (Tfl DNA polymerase) [Akhmetzjanov, A. A., and Vakhitov, V. A. (1992) Nucleic Acids Research 20:5839, GenBank Accession No. X66105], Thermus thermophilus (Tth DNA polymerase) [U.S. Pat. No. 5,618,711] and others, are perhaps the most-used in PCR amplification of DNA and in related DNA primer extension techniques. These enzymes are representative of a family of DNA polymerases like the E. coli DNA polymerase I [Joyce, C. M., and Steitz, T. A. (1994) Annu. Rew. Biochem., 63, 777-822; Steitz, T. A. (1999) J. Biol. Chem., 274, 17395-17398].

Using techniques with an amplification or primer extension step has driven concern for the efficiency and sensitivity of the polymerase used. For enhancing the efficiency of DNA polymerase reactions, which are catalyzed by DNA polymerases, a few approaches were developed.

One way to improving PCR and sequencing reactions is through the use of additives. For example, some reagents like tetramethylammonium (TMA) chloride (Chevet, E., et al, (1995) Nucleic Acids Res., 23, 3343-3344; Hung, T., et al, (1990) Nucleic Acids Res., 18, 4953; Warner, C. K. and Dawson, J. E. (1996) In Persing, D. H. (ed.), PCR Protocols for Emerging Infectious Diseases. ASM Press, Washington D.C.), dimethyl sulfoxide (Winship, P. R. (1989) Nucleic Acids Res., 17, 1266; Bookstein, R., et al, (1990) Nucleic Acids Res., 18, 1666; Sidhu, M. K., et al, (1996) Biotechniques, 21, 44-47.), amides (Chakrabarti, R., et al, (2001) Nucleic Acids Res., 29, 2377-2381) and betaine (Weissensteiner, T., et al, (1996) Biotechniques, 21, 1102-1108; Henke, W., et al, (1997) Nucleic Acids Res., 19, 3957; U.S. Pat. No. 6,270,962) are capable of improving the efficacy and specificity of PCR. These reagents are often used as components of the commercial optimization and enhancer kits for PCR.

Another way to improving PCR is through the use of combinations of polymerases comprising a thermostable DNA polymerase lacking 3′-5′ exonuclease activity and a thermostable DNA polymerase exhibiting 3′-5′ exonuclease activity [U.S. Pat. No. 5,436,149 and U.S. Pat. No. 6,410,277]. For example, a number of polymerase combinations were tested by Bames (Proc. Nat. Acad. Sci. USA, 91: 2216-2220 (1994).

Certain proteins can be also used for enhancing DNA polymerase reactions. These accessory proteins can interact with DNA polymerases and improve polymerase activity and/or the processivity of polymerases, and they can be very useful in enhancing polymerase reactions. For example, bacterial thioredoxin combined with T7 DNA polymerase increases processivity of this polymerase. T7 DNA polymerase, the product of the viral gene 5, by itself has low processivity. It dissociates from a primer-template after the incorporation of <15 nt (Tabor, S., Huber, H. E. & Richardson, C. C. (1987) ° J. Biol. Chem. 262, 16212-16223). Upon infection of Escherichia coli, T7 annexes a host protein, thioredoxin, to serve as its processivity factor (Modrich, P. & Richardson, C. C. (1975) J. Biol. Chem. 250, 5515-5522). T7 DNA polymerase and thioredoxin bind in a one-to-one complex with an apparent dissociation constant of 5 nM (Huber, H. E., Russel, M., Model, P. & Richardson, C. C. (1986) J. Biol. Chem. 261, 15006-15012). The binding of thioredoxin to T7 DNA polymerase increases the affinity of the polymerase specifically to a primer-template by 80-fold (Huber, H. E., Tabor, S. & Richardson, C. C. (1987) J. Biol. Chem. 262, 16224-16232). A consequence of the increased affinity for a primer-template is the ability of T7 DNA polymerase to extend a primer on single-stranded DNA (ssDNA) by thousands of nucleotides without dissociating (Tabor, S., Huber, H. E. & Richardson, C. C. (1987) J. Biol. Chem. 262, 16212-16223).

Another example of enhancing a DNA polymerase reaction by accessory proteins is the using cell extracts and protein complexes isolated from archaebacteria Pyrococcus furiosus (Pfu) for improving polymerase activity and processivity of Pfu DNA polymerase [U.S. Pat. No. 6,444,428].

Accordingly, the identification and use of proteins that can interact with bacterial DNA polymerases like E. coli DNA polymerase I and enhance the DNA polymerase reactions as accessory proteins would be useful in a variety of in vitro DNA synthesis applications.

### SUMMARY OF THE INVENTION

The present invention provides a method for improvement of DNA enzymatic synthesis and amplifications. In particular, the invention relates primarily to enhancing the yield of PCR. The methods of the invention are particularly useful for enhancing yield of a long distance PCR and PCR amplification of low-copy DNA template. The invention can also be useful for improving other laboratory procedures using DNA polymerases, such as primer extension and DNA sequencing.

The present invention provides methods, proteins and reaction kits for increasing the yield of products in reactions catalyzed by DNA polymerases. The increase in products of DNA polymerase reactions is achieved by adding DNA ligase protein to the reaction mixture containing DNA polymerase.

The DNA polymerases that are used for performing the DNA polymerase reactions can be representatives of a family of DNA polymerases like E. coli DNA polymerase I [Joyce, C. M., and Steitz, T. A. (1994) Annu. Rew. Biochem., 63, 777-822; Steitz, T. A. (1999) J. Biol. Chem., 274, 17395-17398]. The DNA ligases that are used for enhancing the DNA polymerase reactions can be bacterial DNA ligases.

The invention provides methods for enhancing DNA polymerase reactions by the addition of a DNA ligase protein, such as protein of NAD-dependent DNA ligase from Thermus aquaticus (Taq DNA ligase), Thermus thermophilus (Tth DNA ligase), Thermus flavus (Tfl DNA ligase) or E. coli (E. coli DNA ligase), to the reaction mixture containing a DNA polymerase like E. coli DNA polymerase I, such as Taq DNA polymerase, Tth DNA polymerase, Tfl DNA polymerase or E. coli DNA polymerase I. According to the invention in particular bacterial DNA ligase is applied to enhance bacterial DNA polymerase activity.

In particular, the invention provides a method for enhancing a DNA polymerase reaction by including in the reaction a mixture containing Taq or Tth DNA polymerase, a protein of bacterial DNA ligase from Thermus aquaticus (Taq DNA ligase) or Thermus thermophilus (Tth DNA ligase). In a certain embodiment of the invention the mixture contains at least a DNA polymerase lacking 3′-5′ exonuclease activity and a DNA polymerase exhibiting a 3′-5′ exonuclease activity, or any other mixture of at least two DNA polymerase activity.

The present invention allows improving the efficacy of DNA polymerase reactions, such as primer extension reaction, DNA sequencing, nick-translation, reverse transcription, PCR, and particularly long distance PCR and PCR amplification of low-copy DNA template.

The compositions, reaction mixtures and kits of the invention contain DNA ligase proteins, which are used to improve enhance the efficacy of DNA polymerase reactions. Furthermore, the compositions, reaction mixtures and kits may contain a plurality of additional reaction components. Among the additional reaction components, one may include an enzyme such as a DNA polymerase.

Other features, aspects and advantages of the invention will be, or will become, apparent to one with skill in the art upon examination of the following figures and detailed description. It is intended that all such additional systems, features, aspects and advantages included within this description are within the scope of the invention, and are protected by the following claims.

### BRIEF DESCRIPTION OF THE FIGURES

These and other features, aspects, and advantages of the present invention will become better understood with regard to the following description, claims and accompanying drawings, where:

FIG. 1 depicts an electrophoretic analysis of the PCR products obtained in Example 1. A 10,000-bp DNA fragment was amplified from 7.5 ng of phage λ genomic DNA in 32 cycles. The PCR was performed with 2.5 U Taq DNA polymerase without extra additives (lane 1) and in presence of 12.5 U, 25 U, 37.5 U, 50 U Taq DNA ligase (lanes 2, 3, 4, 5).

FIG. 2 depicts an electrophoretic analysis of the PCR products obtained in Example 2. A 10,000-bp DNA fragment was amplified from 7.5 ng of phage λ genomic DNA in 32 cycles. The PCR was performed with 2.5 U Taq DNA polymerase without extra additives (lane 1) and in presence of 12.5 U, 25 U, 37.5 U, 50 U Tth DNA ligase (lanes 2, 3, 4, 5).

FIG. 3 depicts an electrophoretic analysis of the PCR products obtained in Example 3. A 15,000-bp DNA fragment was amplified from 7.5 ng of phage λ genomic DNA in 32 cycles. The PCR was performed with 2.5 U Tth DNA polymerase without extra additives (lane 1) and in presence of 12.5 U, 25 U, 37.5 U, 50 U Tth DNA ligase (lanes 2, 3, 4, 5).

FIGS. 4, 5 and 6 depict electrophoretic analyses of the PCR products obtained in Example 4.

FIG. 4 depicts electrophoretic analysis of 20,000-bp PCR product amplified from 7.5 ng of phage λ genomic DNA in 32 cycles. The PCR was performed with 2.5 U TripleMaster® Enzyme Mix without extra additives (lane 1) and in presence of 12.5 U, 25 U, 37.5 U, 50 U Tth DNA ligase (lanes 2, 3, 4, 5).

FIG. 5 depicts electrophoretic analysis of 30,000-bp PCR product amplified from 6 ng of phage λ genomic DNA in 32 cycles. PCR was performed with 5 U TripleMaster® Enzyme Mix without extra additives (lane 1) and in presence of 100 U Tth DNA ligase (lane 2).

FIG. 6 depicts electrophoretic analysis of 40,000-bp PCR product amplified from 15 ng of phage λ genomic DNA in 32 cycles. PCR was performed with 5 U TripleMaster® Enzyme Mix without extra additives (lane 1) and in presence of 100 U Tth DNA ligase (lane 2).

FIG. 7 depicts an electrophoretic analysis of the PCR products obtained in Example 5. A 795-bp DNA fragment of CSF3R (colony stimulating factor 3 receptor) gene was amplified from 6 ng of Human genomic DNA in 45 cycles. PCR was performed under conventional conditions with 1 U Taq DNA polymerase without extra additives (lane 1) and in presence of 20 U Tth DNA ligase (lane 2).

FIG. 8 depicts an electrophoretic analysis of the RT-PCR products obtained in Example 6. A 221-bp cDNA fragment of Elongation Factor 1-alpha mRNA of Xenopus laevis embryo was amplified by RT-PCR from 50 ng of total mRNA of Xenopus laevis embryo. Reverse transcription (RT) and PCR reactions were performed with 2 U Tth DNA polymerase without extra additives (lane 1) and in presence of 40 U Tth DNA ligase (lane 2).

### DETAILED DESCRIPTION OF THE INVENTION

In order to provide a clear and consistent understanding of the specification and claims, the following definitions are provided.

Abbreviations: bp=base pairs; kb=kilobase (1000 base pairs); dNTPs=deoxyribonucleoside triphosphates; NAD=β-nicotinamide adenine dinucleotide; RT=reverse transcription; PCR=polymerase chain reaction.

The following 3-letter abbreviations often refer to the enzymes elaborated by the microorganism: Taq=Thermus aquaticus; Tth=Thermus thermophilus; Tfl=Thermus flavus; Tli=Thermococcus literalis; Pfu=Pyrococcus furiosus; Pwo=Pyrococcus woesii.

Terms “thermostable” or “thermally stable” are used interchangeably herein to describe enzymes which can withstand temperatures up to at least 95° C. for several minutes without becoming irreversibly denatured. Typically, such enzymes have an optimum temperature above 45° C., preferably between 50° and 75° C.

The term “modification” of an enzyme as used herein refers to a chemical or genetic modification of enzyme

The term “nucleic acid sequence” or “polynucleotide sequence” refers to a single- or double-stranded polymer of deoxyribonucleotide or ribonucleotide bases read from the 5′ to the 3′ end.

The terms “oligonucleotide primer”, “oligonucleotide” or “primer” refer to a single-stranded polymer of deoxyribonucleotides or ribonucleotides.

The term “complementary” as used herein refers to a relationship between two nucleic acid sequences. One nucleic acid sequence is complementary to a second nucleic acid sequence if it is capable of forming a duplex with the second nucleic acid, wherein each residue of the duplex forms a guanosine-cytidine (G-C) or adenosine-thymidine (A-T) base pair or an equivalent base pair. Equivalent base pairs can include nucleoside or nucleotide analogues other than guanosine, cytidine, adenosine, or thymidine.

The terms “DNA template”, “RNA template” or “template” as used herein, refer to a nucleic acid that is used by a DNA polymerase to synthesize a new complementary nucleic acid.

The term “DNA polymerase” refers to all proteins or peptides exhibiting a DNA polymerase activity, including allelic variants, fragments, derivatives or analogues of naturally occurring, recombinant or synthetic DNA polymerases, either of bacterial or eucaryotic origin.

The term “DNA ligase” refers to all proteins or peptides exhibiting a DNA ligase activity either of synthetic, recombinant or natural origin. Furthermore the term comprises allelic variants, fragments, derivatives or analogues with at least 70%, preferably 80%, most preferred at least 90%, 95%, or 98% identity to one of the DNA ligase from the group of E. coli” DNA ligase or DNA ligases from thermophilic bacteria. Such as from the genus Thermus, e.g. (ligases from T. aquaticus, T. thermophilus, T. rubber, T. filiformis, T. brockianus, T. flavus and T. scotoductus) or fragments thereof.

The term “DNA polymerase reaction” refers to all reactions comprising a DNA polymerase activity.

The present invention provides methods, proteins and reaction kits which allow improving the efficacy of DNA polymerase reactions, such as PCR, primer extension and DNA sequencing. The invention relates primarily to the improvement of PCR and particularly to enhancing yield of a long distance PCR and PCR amplification of low-copy DNA template.

The present invention provides a method for enhancing enzymatic DNA polymerase reactions. This method is based on the fact, which is disclosed in the invention, that a bacterial DNA ligase can interact with a bacterial DNA polymerase (conceivably as an accessory protein) and enhance the efficacy of a DNA polymerase reaction.

The enhancement of a DNA polymerase reaction and the increase in the product of the reaction can be achieved by the addition of a bacterial DNA ligase protein to the reaction mixture containing a bacterial DNA polymerase. The enhancement of the efficacy of DNA polymerase reaction can also be achieved by using a composition comprising a mixture of bacterial DNA ligase and polymerase.

The DNA ligases, which are capable of improving the efficacy and specificity of DNA polymerase reactions, can be NAD-dependent bacterial DNA ligases like E. coli DNA ligase.

The DNA polymerases, which are used for performing the DNA polymerase reactions, can be bacterial polymerases of a family of DNA polymerases like E. coli DNA polymerase I [Joyce, C. M., and Steitz, T. A. (1994) Annu. Rew. Biochem., 63, 777-822; Steitz, T. A. (1999) J. Biol. Chem., 274, 17395-17398].

The enzymatic DNA polymerase reactions, which may be improved by the methods of the invention, can be primer extension reactions, reverse-transcription reactions, DNA sequencing, nick-translation, PCR and other reactions, which can be catalyzed by DNA polymerases.

In an embodiment of the present invention, the DNA polymerase may be selected from the family of DNA polymerases like E. coli DNA polymerase I, such as E. coli DNA polymerase I, Taq DNA polymerase, Tth DNA polymerase, Tfl DNA polymerase and others. This polymerase may be of wild-type sequences or synthetic variants and fragments.

DNA polymerase for use in the present invention may be selected from modified DNA polymerases of the family of DNA polymerases like E. coli DNA polymerase I, e.g. N-terminal deletions of the DNA polymerases, such as Klenow fragment of E. coli DNA polymerase I, N-terminal deletions of Taq polymerase (including the Stoffel fragment of Taq DNA polymerase, Klentaq-235, and Klentaq-278) and others.

Preferred DNA polymerases for use in the invention, include, but are not limited to thermostable DNA polymerases.

Thermostable polymerases may be isolated from thermophilic bacterial sources (e.g., thermophilic genus Thermus) or they may be isolated and prepared by recombinant means. Representative species of the Thermus genus include T. aquaticus, T. thermophilus, T. rubber, T. filiformis, T. brockianus, T. flavus and T. scotoductus.

Examples of thermostable DNA polymerases for use in the present invention, include, but are not limited to: Tth DNA polymerase, Tfl DNA polymerase, Taq DNA polymerase, N-terminal deletions of Taq polymerase (e.g. Stoffel fragment of DNA polymerase, Klentaq-235, and Klentaq-278). Other DNA polymerases include KIenTaq¹, Taquenase™ (Amersham), AdvanTaq™ (Clontech), GoTaq and GoTaq Flexi (Promega).

In another embodiment of the invention, the DNA polymerase can be included in a mixture of enzymes for performing a DNA polymerase reaction. In a preferred embodiment, the mixture comprises at least one DNA polymerases from the family of DNA polymerases like E. coli DNA polymerase I lacking 3′-5′ exonuclease activity and at least one DNA polymerase exhibiting 3′-5′ exonuclease activity.

Examples of the DNA polymerases lacking 3′-5′ exonuclease activity, include, but are not limited to Taq DNA polymerase, Tth DNA polymerase, Tfl DNA polymerase, Klenow (exo-) fragment of E. coli DNA polymerase I, N-terminal deletions of Taq polymerase (including the Stoffel fragment of DNA polymerase, Klentaq-235, and Klentaq-278) and others.

Examples of DNA polymerases exhibiting 3′-5′ exonuclease activity, include, but are not limited to E. coli DNA polymerase I, Klenow (exo+) fragment of E. coli DNA polymerase I, T4 DNA polymerase, Pyrococcus furiosus (Pfu) DNA polymerase, Thermotoga maritima (Tma) DNA polymerase, Thermococcus litoralis (Tli) DNA polymerase (also referred to as Vent_{R}®), Pyrococus GB-D DNA polymerase, Pyrococus kodakaraensis (KOD) DNA polymerase, Pfx, Pwo, and DeepVent_{R}® polymerases.

Examples of DNA polymerase mixtures for use in the invention include, but are not limited to, mixtures disclosed in e.g., U.S. Pat. Nos. 5,436,149 and 6,410,277.

Preferred mixtures of DNA polymerases, for use in the invention, comprise thermostable DNA polymerases.

Commercially available DNA polymerase mixtures for use in the invention include, but are not limited to, TaqLA, TthLA or Expand High Fidelity^{plus} Enzyme Blend (Roche); TthXL KlenTaqLA, (Perkin-Elmer); ExTaq® (Takara Shuzo); Elongase® (Life Technologies); Advantaget™ KlenTaq, Advantage™ Tth and Advantage2™ (Clontech); TaqExtender™ (Stratagene); Expand™ Long Template and Expand™ High Fidelity (Boehringer Mannheim); and TripleMaster™ Enzyme Mix (Eppendorf).

In an embodiment of the invention, a protein, which is used for enhancing a DNA polymerase reaction, is a bacterial DNA ligase protein.

In a preferred embodiment of the invention, said bacterial DNA ligase can be a NAD-dependent DNA ligase like E. coli DNA ligase. In particular, preferred DNA ligase proteins for use in the invention, include, but are not limited to E. coli DNA ligase and thermostable DNA ligases from thermophilic bacterial sources, such as thermophilic genus Thermus, e.g. ligases from T. aquaticus, T. thermophilus, T. rubber, T. filiformis, T brockianus, T. flavus and T. scotoductus.

DNA ligase proteins, for use in the present invention, may be of wild-type sequences or synthetic variants and fragments. These proteins may be isolated from bacterial or eucaryotic sources or they may be isolated and prepared by recombinant means.

DNA ligase proteins or synthetic variants and fragments of them, for use in the invention, may exhibit or not exhibit DNA ligase activity. For example, a NAD-dependent DNA ligase, such as Taq or Tth DNA ligase, needs NAD for exhibiting DNA ligase activity, but, as it is shown in the Examples, Taq DNA ligase and Tth DNA ligase are able to enhance DNA polymerase reactions without NAD, which presence is not necessary. Thus, the exhibiting DNA ligase activity is not necessary for enhancing DNA polymerase reactions by the DNA ligase proteins.

In an embodiment of the invention, the enhancing a DNA polymerase reaction and the increase in a product of the reaction can be achieved by the addition of said DNA ligase protein (one or more), to the reaction mixture containing at least one DNA polymerase like E. coli DNA polymerase I. In another embodiment of the invention, the enhancing DNA polymerase reaction can also be achieved by the using a composition comprising a mixture of said DNA ligase and polymerase.

The suitable combinations of the DNA ligases and polymerases, for the use in the invention, include, but are not limited to combinations of E. coli DNA ligase and E. coli DNA polymerase I; and combinations of thermostable DNA ligases and polymerases from genus Thermus. The preferred combinations of DNA ligases and polymerases can include, but are not limited to: Taq DNA ligase and Taq DNA polymerase, or Tth DNA polymerase, or Tfl DNA polymerase; Tth DNA ligase and Taq DNA polymerase, or Tth DNA polymerase, or Tfl DNA polymerase; Tfl DNA ligase and Taq DNA polymerase, or Tth DNA polymerase, or Tfl DNA polymerase).

DNA ligases and DNA polymerases, which can be used in the invention, can be added to the reaction mixture separately or together, as components of the compositions.

The compositions, reaction mixtures and kits of the invention may comprise said DNA ligases (one or more) or said combinations of the DNA ligases and polymerases (one or more). Furthermore the compositions, reaction mixtures and kits may contain a plurality of additional reaction components. The additional reaction components may be enzymes, proteins and chemical compounds, such as template nucleic acid(s), oligonucleotide primer(s), dNTPs and others. Preferred additional enzymes may be inorganic Pyrophosphatases (PPase) and DNA polymerases exhibiting 3′-5′ exonuclease activity, particularly, thermostable enzymes, such as Tth PPase or Taq PPase and DNA polymerases: Pfu, Tma, Tli, Pfx, Pwo, KOD, Ventᵣ® and DeepVent_{R}®.

In one aspect, the present invention provides a reaction kit for increasing the efficacy of DNA polymerase reactions. The kit may include a DNA ligase (or ligases), or a composition containing this protein. The kit may further include one or more additional reaction components to facilitate the enzymatic process. The kit may further include one or more DNA polymerases for performing the enzymatic process.

Generally, a kit may comprise a first container containing a DNA ligase or a composition containing this protein and at least a second container having one or more components suitable for performing a DNA polymerase reaction. The second container may contain one of more of (a) dNTPs; (b) ddNTPs; (c) a DNA polymerase; (d) reaction buffer(s) and (e) a primer. The kit may contain two or more, e.g. three, four or five separate containers with these or other components packaged separately or in combinations thereof. Kits may also contain instructions for use of the reagents.

The present invention allows improving the efficacy of DNA polymerase reactions, such as primer extension reaction, DNA sequencing, nick-translation, reverse-transcription (RT), PCR, RT-PCR and other reactions, which can be catalyzed by a DNA polymerase like E. coli DNA polymerase I.

In a preferred embodiment, the present invention includes proteins and methods for increasing the efficacy of PCR and RT-PCR. Specifically, the present invention provides processes and kits for performing a long distance PCR and PCR amplification of low-copy DNA template. The processes and kits utilize the step of addition of the DNA ligase proteins, which are capable of improving the PCR efficiency and described herein, to the reaction mixture of PCR.

Preferred DNA polymerases for use in PCR applications include thermally stable DNA polymerases and/or combinations thereof. Thermally stable DNA polymerases may include but are not limited to those mentioned herein above.

Preferred DNA ligases for enhancing DNA polymerase reaction in PCR applications include thermally stable DNA ligases and/or combinations thereof. Thermally stable DNA ligases may include but are not limited to those mentioned herein above.

Preferred compositions, reaction mixtures and kits of the invention, for use in PCR applications, include combinations of thermostable DNA ligases and polymerases. Combinations of thermostable DNA ligases and polymerases may include, but are not limited to those mentioned herein above.

The addition of the ligase proteins described herein facilitates the enhancing of PCR. See examples herein, for a demonstration of the effects of Taq DNA ligase and Tth DNA ligase on efficacy of PCR performed using Taq or Tth polymerases. Other DNA ligase proteins of the invention can be used in combinations (described herein above) with DNA polymerases in a similar manner to improve efficacy of PCR or other DNA polymerase reactions.

The following Examples illustrate aspects of the invention. The Examples are illustrative of, but not binding on, the present invention. Any methods, preparations, solutions and such like, which are not specifically defined, may be found in Sambrook et al [Sambrook et al., (1989) “Molecular cloning—A Laboratory Manual”, Cold Spring Harbor Laboratory Press]. All solutions are aqueous and made up in sterile, deionised water, unless otherwise specified. Taq and Tth DNA polymerases were obtained from Roche Molecular Systems, Inc. The TripleMaster® Enzyme Mix for PCR, which comprises a mixture of Taq DNA polymerase and a proofreading DNA polymerase exhibiting 3′-5′ exonuclease activity, was obtained from Eppendorf. Taq DNA ligase was obtained from New England Biolabs, Inc. Tth DNA ligase was obtained by the method described in by Barany, F. and Gelfand, D. H. [(1991), Gene, 109, 1-11]. Other reagents were obtained from GeneCraft (Germany).

### EXAMPLES

### Example 1

### Enhancing the Yield of a Long-Distance PCR Performed with Taq DNA Polymerase by Adding Taq DNA Ligase to the Reaction Mixture

A 10,000-bp DNA fragment was amplified from 7.5 ng of phage λ genomic DNA in 32 cycles: 93° C.-45 sec; 58° C.-45 sec; 70° C.-8 min. The reaction mixture (50 μl) contained: 2.5 mM MgCl₂, 20 mM Tris-HCl (pH 9.0 at 25° C.), 50 mM KCl, 0.1% Triton X-100, 0.5 mM each dNTP, 20 pmol primer Pr1 (5′-ctgatcagttcgtgtccgtacaactggcgtaatc), 20 pmol primer Pr2 (5′-atacgctgtattcagcaacaccgtcaggaacacg), and 2.5 U Taq DNA polymerase.

PCR reactions were performed in the absence of Taq DNA ligase and in the presence of Taq DNA ligase. Taq DNA ligase was added to the reaction mixtures in amounts corresponding to 12.5 U, 25 U, 37.5 U and 50 U. (One unit is defined as the amount of DNA ligase required to give 50% ligation of the 12-base pair cohesive ends of 1 μg of BstE II-digested λ DNA in a total reaction volume of 50 μl in 15 minutes at 45° C.)

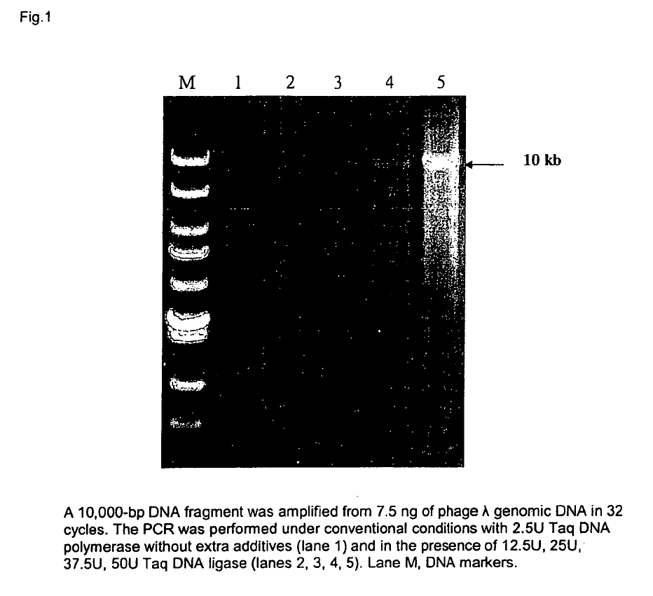

FIG. 1 depicts the electrophoretic analysis of the amplification products obtained. The method of present invention (addition of Taq DNA ligase to the reaction mixture containing Taq polymerase) provided a significant increase of the yield of polymerase reaction. Compared to the conventional PCR procedure without extra additives (lane 1), a detectable amount of the desired product was obtained by adding Taq DNA ligase (note the presence of the target amplification product in lanes 3 through 5 compared to lane 1).

### Example 2

### Enhancing the Yield of a Long-Distance PCR Performed with Taq DNA Polymerase by Adding Tth DNA Ligase to the Reaction Mixture

A 10,000-bp DNA fragment was amplified from 7.5 ng of phage λ genomic DNA in 32 cycles: 93° C.-45 sec; 58° C.-45 sec; 70° C.-8 min. The reaction mixture (50 μl) contained: 2.5 mM MgCl₂, 20 mM Tris-HCl (pH 9.0 at 25° C.), 50 mM KCl, 0.1% Triton X-100, 0.5 mM each dNTP, 20 pmol primer Pr1 (5′-ctgatcagttcgtgtccgtacaactggcgtaatc), 20 pmol primer Pr2 (5′-atacgctgtattcagcaacaccgtcaggaacacg), and 2.5 U Taq DNA polymerase.

PCR reactions were performed in the absence of Tth DNA ligase and in the presence of Tth DNA ligase. Tth DNA ligase was added to the reaction mixtures in amounts corresponding to 12.5 U, 25 U, 37.5 U and 50 U. (One unit is defined as the amount of DNA ligase required to give 50% ligation of the 12-base pair cohesive ends of 1 μg of BstE II-digested λ DNA in a total reaction volume of 50 μl in 15 minutes at 45° C.).

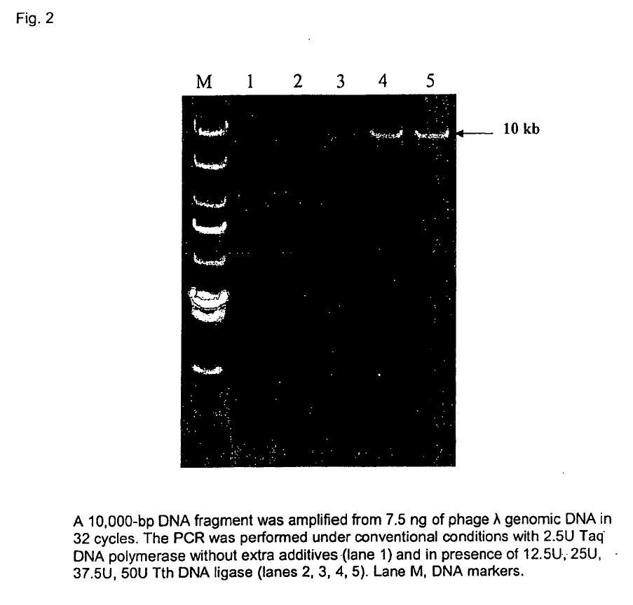

FIG. 2 depicts the electrophoretic analysis of the amplification products obtained. The method of present invention (addition of Tth DNA ligase to the reaction mixture containing Taq polymerase) provided a considerable increase of the yield of polymerase reaction. Compared to the conventional PCR procedure without extra additives (lane 1), increasing amounts of the desired product were obtained by adding Tth DNA ligase (note the increase of the amount of the target amplification product in lanes 2 through 5 compared to lane 1).

### Example 3

### Enhancing the Yield of a Long-Distance PCR Performed with Tth DNA Polymerase by Adding Tth DNA Ligase to the Reaction Mixture

A 15,000-bp DNA fragment was amplified from 7.5 ng of phage λ genomic DNA in 32 cycles: 93° C.-45 sec; 58° C.-45 sec; 70° C.-8 min. The reaction mixture (50 μl) contained: 2.5 mM MgCl₂, 20 mM Tris-HCl (pH 9.0 at 25° C.), 50 mM KCl, 0.1% Triton X-100, 0.5 mM each dNTP, 20 pmol primer Pr1 (5′-ctgatcagttcgtgtccgtacaactggcgtaatc), 20 pmol primer Pr3 (5′-ccagccgcaatatctggcggtgcaatatcggtac), and 2.5 U Tth DNA polymerase.

PCR reactions were performed in the absence of Tth DNA ligase and in the presence of Tth DNA ligase. Tth DNA ligase was added to the reaction mixtures in amounts corresponding to 12.5 U, 25 U, 37.5 U and 50 U. (One unit is defined as the amount of DNA ligase required to give 50% ligation of the 12-base pair cohesive ends of 1 μg of BstE II-digested λ DNA in a total reaction volume of 50 μl in 15 minutes at 45° C.).

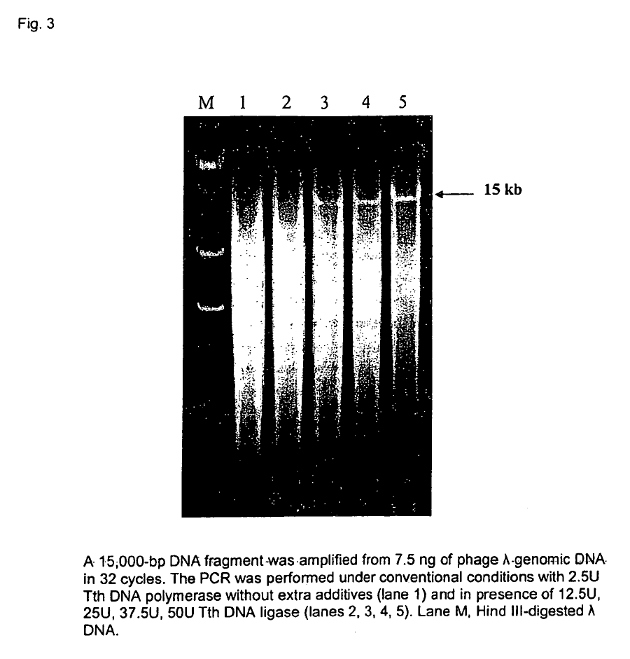

FIG. 3 depicts the electrophoretic analysis of the amplification products obtained. The method of present invention (addition of Tth DNA ligase to the reaction mixture containing Tth polymerase) provided a significant increase of the yield of 15,000-bp target product of polymerase reaction. Compared to the conventional PCR procedure without extra additives (lane 1), detectable amount of the desired product was obtained only by adding Tth DNA ligase (note the presence of the target amplification product in lanes 3 through 5 compared to lane 1).

### Example 4

### Enhancing the Yield of a Long-Distance PCR Performed with Commercially Available Polymerase Mixture “TripleMaster® Enzyme Mix” (Eppendorf) Inclusive of Taq DNA Polymerase and a Proof-Reading DNA Polymerase Exhibiting 3′-5′ Exonuclease Activity, by Adding Tth DNA Ligase to the Reaction Mixture

A 20,000-bp DNA fragment was amplified from 7.5 ng of phage λ genomic DNA in 32 cycles: 93° C.-45 sec; 58° C.-45 sec; 70° C.-10 min. The reaction mixture (50 μl) contained: 2.5 mM MgCl₂, 20 mM Tris-HCl (pH 9.0 at 25° C.), 50 mM KCl, 0.1% Triton X-100, 0.5 mM each dNTP, 20 pmol primer Pr1 (5′-ctgatcagttcgtgtccgtacaactggcgtaatc), 20 pmol primer Pr4 (5′-gtgcaccatgcaacatgaataacagtgggttatc), and 2.5 U of the TripleMaster® Enzyme Mix.

PCR reactions were performed in the absence of Tth DNA ligase and in the presence of Tth DNA ligase. Tth DNA ligase was added to the reaction mixtures in amounts corresponding to 12.5 U, 25 U, 37.5 U and 50 U.

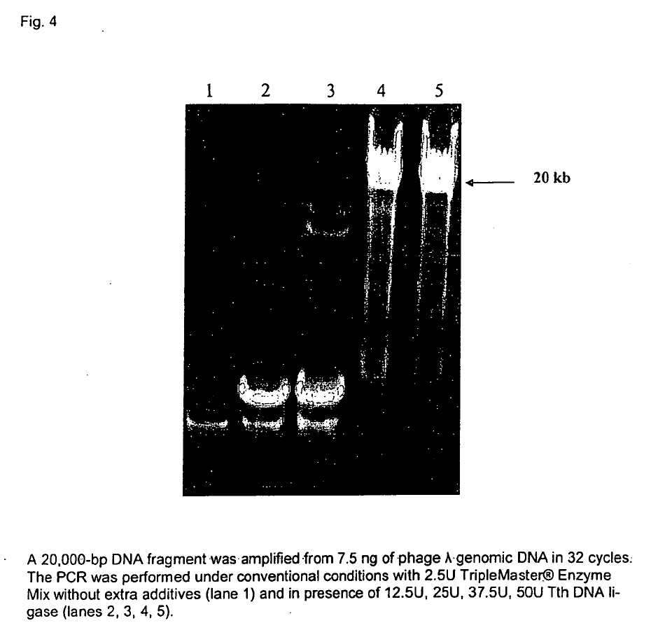

FIG. 4 depicts the electrophoretic analysis of the amplification products obtained. The method of present invention (addition of Tth DNA ligase to the reaction mixture) provided a significant increase of the yield of the target 20,000 bp DNA product. Compared to the conventional PCR procedure without Tth DNA ligase (lane 1), considerable amount of the desired product was obtained only by adding Tth DNA ligase (note the presence of the target amplification product in lanes 4 and 5 compared to lane 1).

A 30,000-bp DNA fragment was amplified from 6 ng of phage λ genomic DNA in 32 cycles: 93° C.-45 sec; 58° C.-45 sec; 70° C.-20 min. The reaction mixture (50 μl) contained: 2.5 mM MgCl₂, 20 mM Tris-HCl (pH 9.0 at 25° C.), 50 mM KCl, 0.1% Triton X-100, 0.5 mM each dNTP, 20 pmol primer Pr1 (5′-ctgatcagttcgtgtccgtacaactggcgtaatc), 20 pmol primer Pr5 (5′-gaaagttatccctagtcagtggcctgaagagac), and 5 U of the TripleMaster® Enzyme Mix.

PCR reactions were performed in the absence of Tth DNA ligase and in the presence of 100 U Tth DNA ligase.

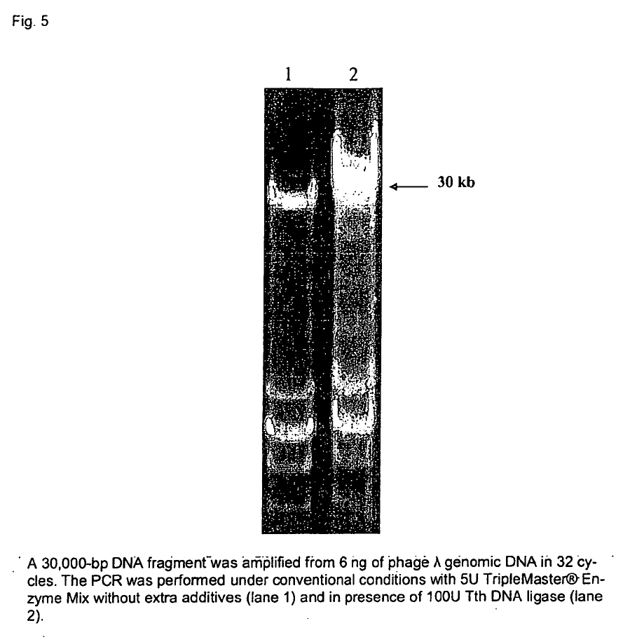

FIG. 5 depicts the electrophoretic analysis of the amplification products obtained. The method of present invention (addition of Tth DNA ligase to the reaction mixture) provided a significant increase of the yield of the target 30,000-bp DNA product. Compared to the conventional PCR procedure without Tth DNA ligase (lane 1), considerable amount of the desired product was obtained by adding Tth DNA ligase (lane 2).

A 40,000-bp DNA fragment was amplified from 15 ng of phage λ genomic DNA in 32 cycles: 93° C.-45 sec; 58° C.-45 sec; 70° C.-20 min. The reaction mixture (50 μl) contained: 2.5 mM MgCl₂, 20 mM Tris-HCl (pH 9.0 at 25° C.), 50 mM KCl, 0.1% Triton X-100, 0.5 mM each dNTP, 20 pmol primer Pr1 (5′-ctgatcagttcgtgtccgtacaactggcgtaatc), 20 pmol primer Pr6 (5′-taatgcaaactacgcgccctcgtatcacatgg), and 5 U of the TripleMaster® Enzyme Mix.

PCR reactions were performed in the absence of Tth DNA ligase and in the presence of 100 U Tth DNA ligase.

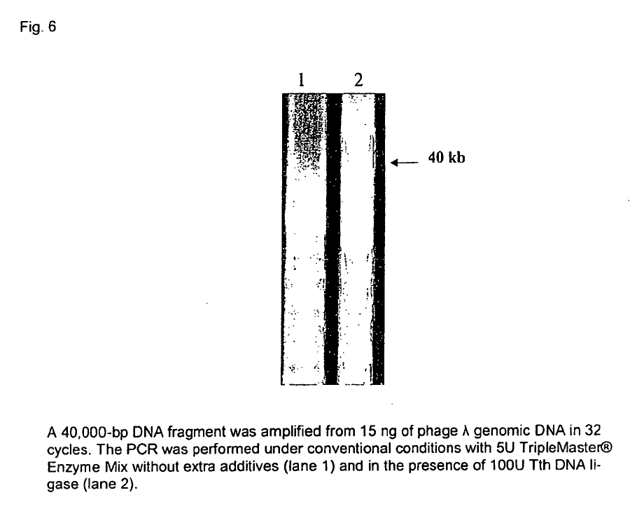

FIG. 6 depicts the electrophoretic analysis of the amplification products obtained. The method of present invention (addition of Tth DNA ligase to the reaction mixture) provided a significant increase of the yield of the target 40,000-bp DNA product. Compared to the conventional PCR procedure without Tth DNA ligase (lane 1), detectable amount of the desired product was obtained by adding Tth DNA ligase (lane 2).

### Example 5

### Enhancing the Yield of a Low Template Copy Number PCR Amplification Performed with Taq DNA Polymerase by Adding Tth DNA Ligase to the Reaction Mixture

A 795-bp DNA fragment of CSF3R (colony stimulating factor 3 receptor) gene was amplified from 6 ng of human genomic DNA (1,000 copies) in 45 cycles: 93° C.-40 sec; 58° C.-40 sec; 72° C.-40 sec. The reaction mixture (50 μl) contained: 2 mM MgCl₂, 20 mM Tris-HCl (pH 9.0 at 25° C.), 50 mM KCl, 0.1% Triton X-100, 0.15 mM each dNTP, 10 pmol primer PrCSFR1 (5′-CCTGGAGCTGAGAACTAC), 10 pmol primer PrCSFR2 (5′-TCCCGGCTGAGTTATAGG), and 1 U Taq DNA polymerase.

PCR reactions were performed in the absence of Tth DNA ligase and in the presence of 20 U Tth DNA ligase.

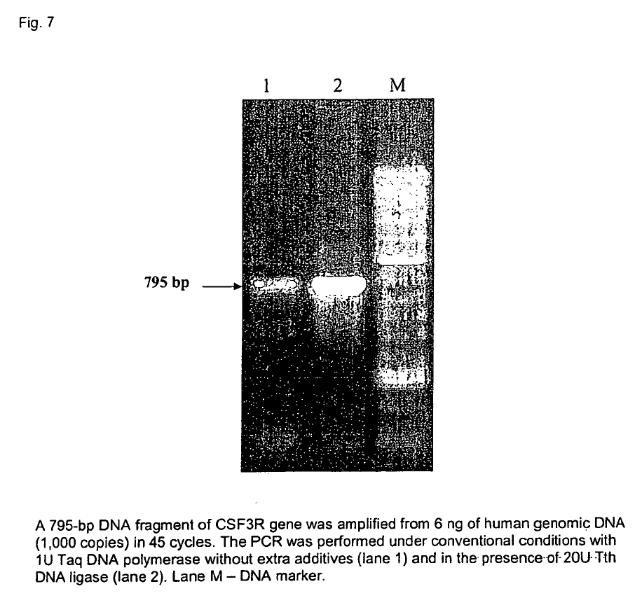

FIG. 7 depicts the electrophoretic analysis of the amplification products obtained. The method of present invention (addition of Tth DNA ligase to the reaction mixture) provided a significant increase of the yield of the target DNA product. Compared to the conventional PCR procedure without Tth DNA ligase (lane 1), a marked increase of the amount of the desired product was obtained by adding Tth DNA ligase to the reaction mixture (lane 2).

### Example 6

### Enhancing the Yield of RT-PCR Performed with Tth DNA Polymerase by Adding Tth DNA Ligase to the Reaction Mixture

A 221-bp cDNA fragment of Elongation Factor 1-alpha mRNA of Xenopus laevis embryo was amplified by RT-PCR from 50 ng of total mRNA of Xenopus laevis embryo. Reverse transcription (RT) was performed with Tth DNA polymerase for 40 min at 58° C. The fragment of cDNA was amplified by PCR in 25 cycles: 93° C.-30 sec; 58° C.-30 sec; 70° C.-30 sec. The reaction mixture for RT-PCR (50 μl) contained: 1 mM MnCl₂, 50 mM Tris-HCl (pH 8.2 at 25° C.), 50 mM KCl, 0.25 mM each dNTP, 15 pmol primer Pr-RT1 (5′-CCTGAACCACCCAGGCCAGATTGGTG), 15 pmol primer Pr-RT2 (5′-GAGGGTAGTCAGAGAAGCTCTCCACG), 2 U Tth DNA polymerase and 50 ng of total mRNA of Xenopus laevis embryo as template.

RT-PCR reactions were performed in the absence of Tth DNA ligase and in the presence of 40 U Tth DNA ligase.

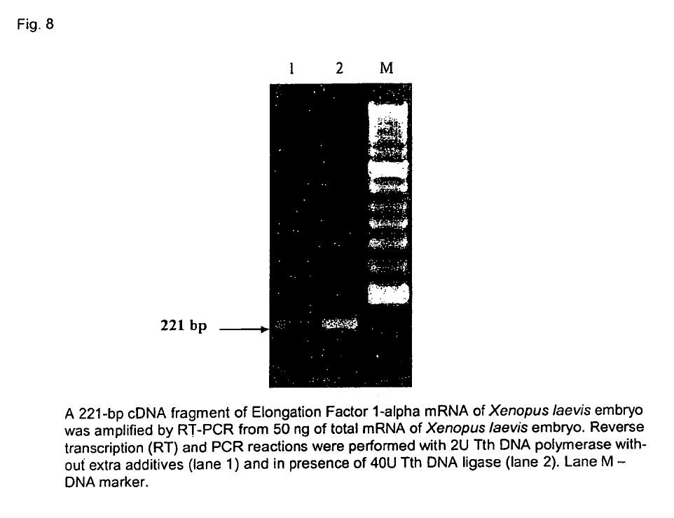

FIG. 8 depicts the electrophoretic analysis of the amplification products obtained. The method of present invention (addition of Tth DNA ligase to the reaction mixture) provided a significant increase of the yield of the target 221-bp cDNA product. Compared to the conventional RT-PCR procedure without Tth DNA ligase (lane 1), a marked increase of the amount of the desired product was obtained by adding Tth DNA ligase to the reaction mixture (lane 2).

1. A method of enhancing a DNA polymerase reaction by including in a reaction mixture containing a DNA polymerase a protein of DNA ligase.

2. The method according to claim 1 wherein said DNA ligase protein is of a wild-type sequence or a synthetic variant.

3. The method according to claim 1 wherein said DNA ligase protein is a protein of NAD-dependent DNA ligase.

4. The method according to claim 1 wherein said DNA ligase protein is a protein of DNA ligase from E. coli (E. coli DNA ligase).

5. The method according to claim 1 wherein said DNA ligase protein is a protein of DNA ligase from Thermus aquaticus (Taq DNA ligase).

6. The method according to claim 1 wherein said DNA ligase protein is a protein of DNA ligase from Thermus thermophilus (Tth DNA ligase).

7. The method according to claim 1 wherein said DNA ligase protein is a protein of DNA ligase from Thermus flavus (Tfl DNA ligase).

8. The method according to claim 1 wherein said DNA ligase protein is a protein of DNA ligase from Thermus rubber.

9. The method according to claim 1 wherein said DNA ligase protein is a protein of DNA ligase from Thermus filiformis.

10. The method according to claim 1 wherein said DNA ligase protein is a protein of DNA ligase from Thermus brockianus.

11. The method according to claim 1 wherein said DNA ligase protein is a protein of DNA ligase from Thermus scotoductus.

12. The method according to claim 1 wherein said DNA polymerase is a DNA polymerase from the family of DNA polymerases like E. coli DNA polymerase I.

13. The method according to claim 1 wherein said DNA polymerase is E. coli DNA polymerase I.

14. The method according to claim 1 wherein said DNA polymerase is the Klenow fragment of E. coli DNA polymerase I.

15. The method according to claim 1 wherein said DNA polymerase is DNA polymerase I from Thermus aquaticus (Taq DNA polymerase).

16. The method according to claim 1 wherein said DNA polymerase is the Stoffel fragment of Taq DNA polymerase.

17. The method according to claim 1 wherein said DNA polymerase is Klentaq DNA polymerase.

18. The method according to claim 1 wherein said DNA polymerase is DNA polymerase I from Thermus thermophilus (Tth DNA polymerase).

19. The method according to claim 1 wherein said DNA polymerase is DNA polymerase I from Thermus flavus (Tfl DNA polymerase).

20. The method according to claim 1 wherein said DNA polymerase is DNA polymerase I from Thermus rubber.

21. The method according to claim 1 wherein said DNA polymerase is DNA polymerase I from Thermus filiformis.

22. The method according to claim 1 wherein said DNA polymerase is DNA polymerase I from Thermus brockianus.

23. The method according to claim 1 wherein said DNA polymerase is DNA polymerase I from Thermus scotoductus.

24. The method of enhancing a DNA polymerase reaction by including in the reaction mixture a protein of DNA ligase according to claim 1, wherein said reaction mixture comprises at least one DNA polymerase lacking 3′-5′ exonuclease activity and at least one DNA polymerase exhibiting 3′-5′ exonuclease activity.

25. The method according to claim 24 wherein said DNA ligase protein is as defined in any one of claims 2 through 11.

26. The method according to claim 24 wherein said DNA polymerase is a DNA polymerase lacking 3′-5′ exonuclease activity from the family of DNA polymerases like E. coli DNA polymerase I.

27. The method according to claim 24 wherein said DNA polymerase lacking 3′-5′ exonuclease activity is as defined in any one of claims 15 through 23.

28. The method according to claim 24 wherein said DNA polymerase exhibiting 3′-5′ exonuclease activity may be selected from the group consisting of E. coli DNA polymerase I, Klenow (exo+) fragment of E. coli DNA polymerase I, T4 DNA polymerase, Pyrococcus furiosus (Pfu) DNA polymerase, Thermotoga maritima (Tma) DNA polymerase, Thermococcus litoralis (Tli) DNA polymerase (also referred to as Vent_{R}®), Pyrococus GB-D DNA polymerase, Pyrococus kodakaraensis (KOD) DNA polymerase, Pfx, Pwo, and DeepVent_{R}® polymerases.

29. The method according to claim 1 wherein said DNA polymerase reaction is used for DNA sequencing.

30. The method according to claim 1 wherein said DNA polymerase reaction is a reaction of nick-translation.

31. The method according to claim 1 wherein said DNA polymerase reaction is a primer extension reaction.

32. The method according to claim 1 wherein said DNA polymerase reaction is a reaction of reverse-transcription (RT).

33. The method according to claim 1 wherein said DNA polymerase reaction is PCR.

34. The method according to claim 1 wherein said DNA polymerase reaction is RT-PCR.

35. A composition of enzymes and proteins, for performing a DNA polymerase reaction by the method defined in claim 1, comprising at least one DNA ligase protein and at least one bacterial DNA polymerase, wherein the composition may comprise one or more additional components.

36. The composition according to claim 35 wherein said bacterial DNA ligase protein is as defined in any one of claims 2 through 11.

37. The composition according to claim 35 wherein said DNA polymerase is as defined in any one of claims 12 through 23.

38. The composition according to claim 35 wherein said DNA ligase protein is E. coli DNA ligase and said DNA polymerase is E. coli DNA polymerase I.

39. The composition according to claim 35 wherein said DNA ligase protein is Taq DNA ligase and said DNA polymerase may be selected from the group consisting of Taq DNA polymerase, Tth DNA polymerase and Tfl DNA polymerase.

40. The composition according to claim 35 wherein said bacterial DNA ligase protein is Tth DNA ligase and said DNA polymerase may be selected from the group consisting of Taq DNA polymerase, Tth DNA polymerase and Tfl DNA polymerase.

41. The composition according to claim 35 wherein said DNA ligase protein is Tfl DNA ligase and said DNA polymerase may be selected from the group consisting of Taq DNA polymerase, Tth DNA polymerase and Tfl DNA polymerase.

42. The composition according to claim 35 wherein said additional component of composition is a DNA polymerase exhibiting 3′-5′ exonuclease activity.

43. The composition according to claim 42 wherein said DNA polymerase exhibiting 3′-5′ exonuclease activity may be selected from the group consisting of E. coli DNA polymerase I, Klenow (exo+) fragment of E. coli DNA polymerase I, T4 DNA polymerase, Pyrococcus furiosus (Pfu) DNA polymerase, Thermotoga maritima (Tma) DNA polymerase, Thermococcus litoralis (Tli) DNA polymerase (also referred to as Vent_{R}®), Pyrococus GB-D DNA polymerase, Pyrococus kodakaraensis (KOD) DNA polymerase, Pfx, Pwo, and DeepVent_{R}® polymerases.

44. The composition according to claim 35 wherein said additional component of composition is an inorganic pyrophosphatase.

45. The composition according to claim 44 wherein said inorganic pyrophosphatase may be selected from the group consisting of E. coli pyrophosphatase, Tth pyrophosphatase, Tfl pyrophosphatase and Taq pyrophosphatase.

46. A kit for performing a DNA polymerase reaction by the method of any one of claims 1 through 34, comprising in separate containers: a) components for DNA polymerase reaction; and b) a container which contains a DNA ligase protein defined in any one of claims 2 through 11, or a composition defined in any one of claims 35 through 45.
